(19) United States
(12) Patent Application Publication
(10) Pub. No.: US 2020/0003741 A1
(43) Pub. Date: Jan. 2, 2020

(54) Sensor Apparatus and Method for Capturing Substances in a Fluid

(71) Applicant: Robert Bosch GmbH, Stuttgart (DE)

(72) Inventors: Thomas Buck, Tamm (DE); Jochen Hoffmann, Renningen (DE)

(21) Appl. No.: 16/487,475

(22) PCT Filed: Feb. 5, 2018
(86) PCT No.: PCT/EP2018/052781

§ 371 Date: Aug. 21, 2019

(30) Foreign Application Priority Data
Feb. 23, 2017 (DE) 10 2017 202 918.8

(51) Int. Cl. G01N 33/00; G01N 1/22; G01N 1/40; G01N 1/42

(52) U.S. Cl. CPC G01N 33/0016; G01N 1/2202; G01N 2033/0019; G01N 1/42; G01N 1/4022

(57) ABSTRACT

A sensor apparatus for capturing substances in a fluid includes a functional element, a vibration generating device, a cooling device, a gas sensor device, and a housing. The functional element is configured to condense and atomize fluid, and the vibration generating device is configured to cause the functional element to mechanically vibrate. The cooling device is configured to cool the functional element, and the gas sensor device is configured to capture gaseous substances in the fluid atomized by the functional element. The housing is configured to accommodate the functional element, the vibration generating device, the cooling device, and the gas sensor device. At least one passage opening for the fluid is formed in the housing.

### PRIOR ART

The invention is based on a device or a method of the category of the independent claims. The subject matter of the present invention is also a computer program.

To identify organic and/or inorganic compounds from a gas phase, a defined gas volume may first of all for example be converted into the liquid phase and accumulated over a defined time interval in order to then convert the condensate back into the gas phase and channel it to a sensing surface in a targeted manner. Conventional systems may have, in addition to the actual gas sensor element, in particular cooling units (for example Peltier elements), microfluidic channel systems (for channeling the gas in a targeted manner), enlarged surfaces such as for example porous materials (in order to offer the gas flowing past an interaction surface that is as large as possible) and heaters (in order to convert the condensate back into the gas phase). To extract liquid contained in gases, molecular sieves, superadsorbers or chemical compounds such as for example hyaluronic acid may also be used.

### DISCLOSURE OF THE INVENTION

Against this background, with the approach presented here, a sensor device, a method, also a controller that uses this method, and finally a corresponding computer program are presented according to the main claims. Advantageous developments and improvements of the device specified in the independent claim are possible by virtue of the measures outlined in the dependent claims.

According to embodiments, integrated detection of chemical compounds or substances is in particular able to be achieved by way of a sensor device that has for example a chip having an in particular pizeoelectrically actuatable functional element whose temperature is able to be lowered by a cooling element, for example a Peltier element. If a fluid flows past such a functional element, precipitation of liquid contained in a gas phase may be induced on the functional element, for example. By virtue in particular of an electrically induced high-frequency oscillation, mechanical energy is then able to be generated in order to convert the condensed liquid back into the gas phase. The functional element and a gas sensor surface may in this case be arranged such that gaseous substances are able to be detected.

Advantageously, according to embodiments, a sensor device with extensive functionality and that is integrated into an extremely small space in a sensor housing is in particular able to be provided. Furthermore, this sensor device may for example offer the possibility of determining a mass of a condensate on the functional element by way of suitable evaluation electronics. In the case of detecting heat-labile substances or target elements, such as for example proteins, human cells, exosomes or hormones, instead of thermal evaporation that could lead to damage of such biological substances, a conversion of a condensate into the gas phase may be achieved at room temperature, as a result of which it is possible to avoid damage to heat-labile substances. In particular no heating element is in this case required to convert a condensate into the gas phase. In particular by virtue of piezoelectric nebulization or atomization, it is possible to save energy, which allows energy-saving operation with regard to use in portable devices. By way of example, high piezoelectric evaporation rates or atomization rates and associated rates or throughputs of up to 10 milliliters per minute may be made possible. In other words, it may be made possible to analyze a large gas volume or large gas volumes. Fluids may also be analyzed. A field of use of the sensor device may thus be further increased. It is furthermore possible to avoid a situation whereby it is necessary to alternate between cooling of a region for the condensate formation and heating of the same region for the conversion of the condensate into the gas phase.

A sensor device for detecting substances in a fluid is presented, wherein the sensor device has at least the following features:

a functional element for condensing and/or atomizing fluid;

an oscillation generator for setting the functional element into mechanical oscillations;

a cooling apparatus for cooling the functional element;

a gas sensor apparatus for detecting gaseous substances in fluid atomized by the functional element; and

a housing for receiving the functional element, the oscillation generator, the cooling apparatus and the gas sensor apparatus, wherein at least one through-aperture for the fluid is formed in the housing.

The substances in the fluid may be at least one substance, at least one molecule, at least one chemical compound or at least one chemical element. The gas sensor apparatus may be configured as a sensor chip or the like. The cooling apparatus may be designed so as to cool the functional element to a dew point temperature of at least one substance to be detected.

According to one embodiment, the functional element may be formed as a beam or as a membrane. In particular, the functional element may be formed as a perforated membrane. If a perforated membrane is used as functional element, condensate formation may be forced on just one side of the membrane. If the membrane is then set in mechanical oscillations or in resonance, the generated drops may be accelerated onto the other side of the membrane. This allows a targeted transfer onto a gas sensor surface of the gas sensor apparatus and may make a phase conversion more effective, which may result in an increased sensitivity of the gas sensor apparatus.

The functional element may also be arranged or able to be arranged so as to be clamped in the housing on one side. Such an embodiment offers the advantage that the functional element is able to be set efficiently and exactly in mechanical oscillations.

The housing may furthermore have a first chamber and a second chamber. In this case, the first chamber and the second chamber may be connected to one another by a connecting aperture. In this case, the functional element, the oscillation generator and the cooling apparatus may be arranged or able to be arranged in the first chamber. The gas sensor apparatus may be arranged or able to be arranged in the second chamber. In other words, a separating wall may be arranged in the housing between the first chamber the second chamber. A situation is thereby able to be achieved whereby the atomized microdroplets do not impinge immediately on a sensor surface of the gas sensor apparatus, but rather first have to cover a certain path in which a phase transition from liquid to gaseous may take place in an accelerated manner before they impinge on the sensor surface, such that more components transition into the gas phase and a sensitivity of the sensor is able to be improved.

The sensor device may additionally have a capillary element. The capillary element may be arranged or able to be arranged spaced apart from the functional element by a capillary intermediate space. The capillary intermediate space may in this case be arranged adjacent to the at least one through-aperture. A fluid is thus able to pass into the capillary intermediate space from the through-aperture due to the capillary effect. Liquids are therefore also able to be analyzed accurately and reliably and in an energy-saving manner by way of the sensor device.

According to one embodiment, two through-apertures for a flow of fluid through the housing may be formed in the housing. In addition or as an alternative, a pressure equalization aperture for pressure equalization may be formed between the housing and an environment. Continuous and energy-saving sensing of gas or of a liquid may be made possible.

The gas sensor apparatus and the cooling apparatus may also be formed in a common substrate and additionally or alternatively on a common substrate. The common substrate may be configured as a chip substrate. The common substrate may furthermore have an electric circuit, in particular an integrated circuit, for example an application-specific integrated circuit. Such an embodiment offers the advantage of being space-saving, easy to manufacture and inexpensive.

The functional element, the oscillation generator and the cooling apparatus may in particular be arranged or able to be arranged in the form of a stack. The functional element is thereby able to be cooled effectively and in a space-saving manner and set into mechanical oscillations.

A method for detecting substances in a fluid is also presented, wherein the method is able to be executed in connection with one embodiment of a sensor device described here, wherein the method has at least the following steps:

actuating the cooling apparatus so as to cool the functional element in order to condense fluid on the functional element;

exciting the oscillation generator so as to set the functional element into mechanical oscillations in order to atomize condensed fluid; and

operating the gas sensor apparatus so as to detect gaseous substances in fluid atomized by the functional element.

This method may be implemented for example in software or hardware or in a mixed form of software and hardware, for example in a controller. The method may be executed using one embodiment of the sensor device mentioned above. In the actuation step, an actuation signal may be applied to the cooling apparatus. In the excitation step, an excitation signal may be applied to the oscillation generator. In the operating step, a sensor signal may be received or read by the gas sensor apparatus.

According to one embodiment, in the excitation step, the oscillation generator may be excited so as, in a first quantitative detection mode of operation, to set the functional element into mechanical oscillations at a first amplitude and additionally or alternatively at a first frequency and so as, in a second qualitative detection mode of operation, to set the functional element into mechanical oscillations at a second amplitude and additionally or alternatively at a second frequency. In this case, the first amplitude may be less than the second amplitude. The first frequency and the second frequency may be the same or different. In the first mode of operation, a mass determination of the condensate on the functional element may thus be made possible, wherein a mass accumulation on the condensate trap of the sensor is able to be measured continuously without extra structural expenditure. The values detected by the gas sensor may thus be detected in a more quantitative manner, since it is possible to produce a connection between the analyzed air volume and the moisture contained therein.

The method may also have a step of conveying a fluid flow into the housing of the sensor device and additionally or alternatively through the housing of the sensor device. Such an embodiment offers the advantage that an accuracy of a quantitative detection is able to be further increased, since a defined volume flow of fluid is able to be supplied.

The approach presented here furthermore provides a controller that is designed to perform, actuate or implement the steps of a variant of a method presented here in corresponding apparatuses. The object on which the invention is based is also able to be achieved quickly and efficiently by virtue of this embodiment variant of the invention in the form of a controller.

To this end, the controller may have at least one computer unit for processing signals or data, at least one storage unit for storing signals or data, at least one interface to a sensor or an actuator for reading sensor signals from the sensor or for outputting control signals to the actuator and/or at least one communication interface for reading or outputting data that are embedded into a communication protocol. The computer unit may be for example a signal processor, a microcontroller or the like, wherein the storage unit may be a flash memory, an EEPROM or a magnetic storage unit. The communication interface may be designed to read or output data wirelessly and/or in a wired manner, wherein a communication interface that is able to read or output wired data is able to read these data for example electrically or optically from a corresponding data transmission line or output them into a corresponding data transmission line.

A controller may in this case be understood to mean an electrical device that processes sensor signals and outputs control signals and/or data signals depending thereon. The controller may have an interface that may be designed in the form of hardware and/or software. In the case of a design in the form of hardware, the interfaces may be for example part of what is known as a system ASIC that contains a wide variety of functions of the controller. It is however also possible for the interfaces to be dedicated integrated circuits or to consist at least partly of discrete components. In the case of a design in the form of software, the interfaces may be software modules that are present for example in a microcontroller in addition to other software modules.

In one advantageous configuration, the controller controls a sensor device for fluid, in particular controls an oscillation generator and a cooling apparatus of the sensor device. Piezoelectric elements and also Peltier elements may be actuated in this case. The controller may furthermore access sensor signals from the gas sensor apparatus, for example.

A computer program product or computer program containing program code that may be stored on a machine-readable carrier or storage medium, such as a semiconductor memory, a hard disk drive or an optical memory and is used to perform, implement and/or actuate the steps of the method according to one of the embodiments described above, in particular when the program product or program is executed on a computer or a device, is also advantageous.

Exemplary embodiments of the approach presented here are illustrated in the drawings and explained in more detail in the following description, in which drawings:

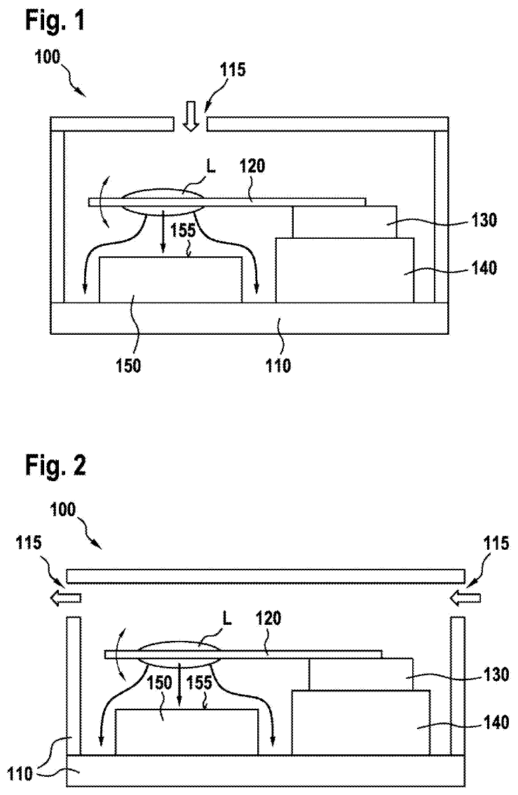

FIG. 1 shows a schematic sectional illustration of a sensor device according to one exemplary embodiment;

FIG. 2 shows a schematic sectional illustration of a sensor device according to one exemplary embodiment;

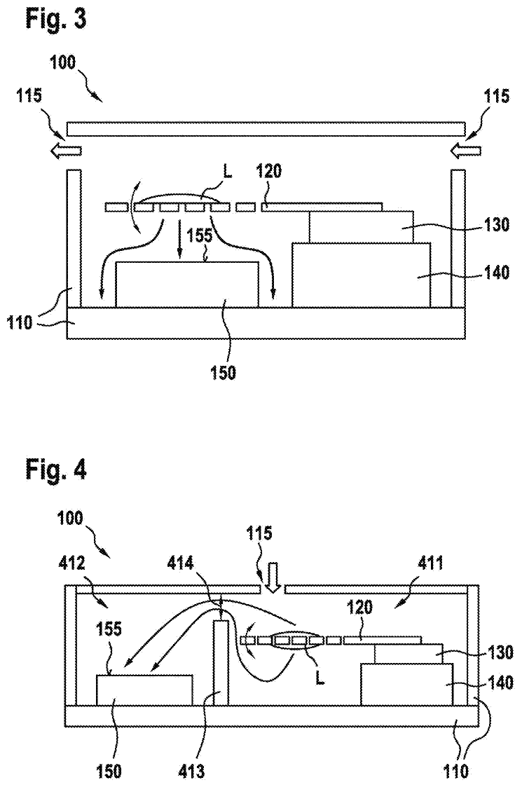

FIG. 3 shows a schematic sectional illustration of a sensor device according to one exemplary embodiment;

FIG. 4 shows a schematic sectional illustration of a sensor device according to one exemplary embodiment;

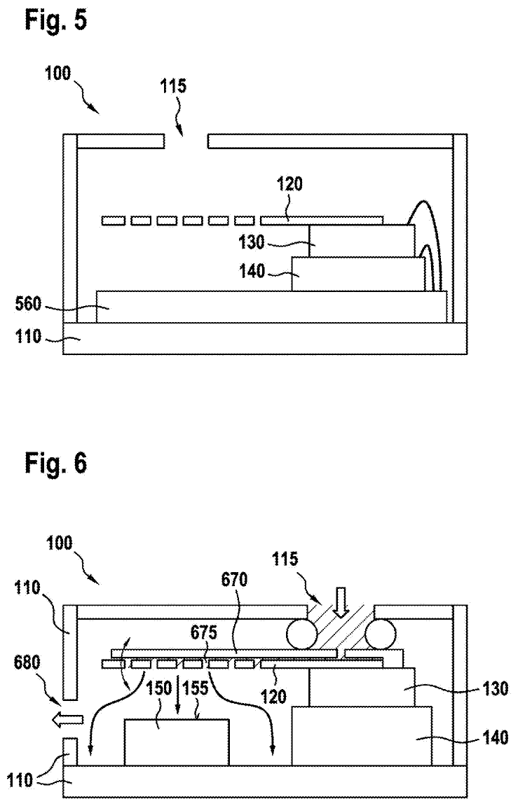

FIG. 5 shows a schematic sectional illustration of a sensor device according to one exemplary embodiment;

FIG. 6 shows a schematic sectional illustration of a sensor device according to one exemplary embodiment;

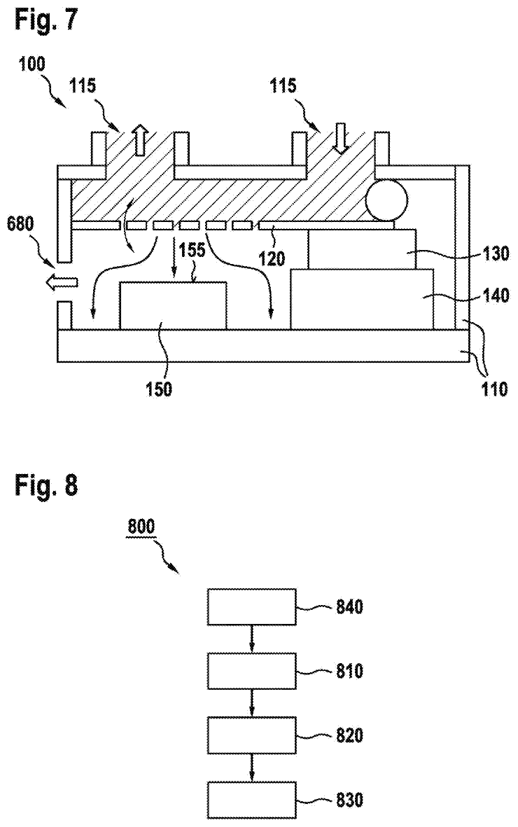

FIG. 7 shows a schematic sectional illustration of a sensor device according to one exemplary embodiment; and

FIG. 8 shows a flowchart of a detection method according to one exemplary embodiment.

In the following description of expedient exemplary embodiments of the present invention, the same or similar reference signs are used for the elements that are illustrated in the different figures and that have a similar effect, a repeated description of these elements being dispensed with.

FIG. 1 shows a schematic sectional illustration of a sensor device 100 according to one exemplary embodiment. The sensor device 100 is designed to detect substances in a fluid. The fluid may have at least one liquid, at least one gas or a gas-liquid mixture. The substances to be detected may represent at least one chemical compound, at least one chemical element and/or at least one molecule.

The sensor device 100 has a housing 110. By way of example, just one through-aperture 115 for the fluid is formed in the housing 110 according to the exemplary embodiment illustrated in FIG. 1. The housing 110 is thus open to the fluid in the region of the through-aperture 115. A functional element 120, an oscillation generator 130, a cooling apparatus 140 and a gas sensor apparatus 150 having a gas sensor surface 155 are received or arranged in the housing 110.

According to the exemplary embodiment shown in FIG. 1, the functional element 120, the oscillation generator 130 and the cooling apparatus 140 are arranged in the form of a stack or stacked on top of one another. In this case, the oscillation generator 130 is arranged between the functional element 120 and the cooling apparatus 140. The gas sensor apparatus 150 is arranged spaced apart from the stack. In this case, the gas sensor surface 155 faces the functional element 120 or a partial section of the functional element 120.

The functional element 120 is intended to condense and/or atomize fluid. According to the exemplary embodiment illustrated in FIG. 1, the functional element 120 is formed as a beam. More precisely, the functional element 120 is in this case formed as a beam that is clamped on one side or arranged in the housing 110 so as to be clamped on one side. In this case, a first partial section of the functional element 120 is applied to the oscillation generator 130, wherein a second partial section of the functional element 120 overhangs. The second partial section of the functional element 120 extends over the gas sensor apparatus 150. A main surface of the second partial section of the functional element 120 faces the gas sensor surface 155 of the gas sensor apparatus 150.

The oscillation generator 130 is designed to set the functional element 120 into mechanical oscillations. In this case, the oscillation generator 130 is for example configured as an ultrasound generator, a piezoelectric element or the like.

The cooling apparatus 140 is designed to cool the functional element 120. In this case, the cooling apparatus 140 is in particular designed to cool the functional element 120 to a dew point temperature of a substance to be detected in the fluid. The cooling apparatus 140 is configured for example as a Peltier element or the like.

The gas sensor apparatus 150 is designed to detect gaseous substances or substances that have transitioned back into the gas phase in fluid that has condensed on the functional element 120 and has been atomized by the functional element 120. In this case, the gas sensor apparatus 150 is configured for example as a gas sensor chip having the gas sensor surface 155.

In other words, FIG. 1 shows a schematic cross-section through the sensor device 100. The functional element 120 in the form of a beam is connected frictionally to the oscillation generator 130 configured as an ultrasound generator or piezoelectric element. The oscillation generator 130 is in a thermally conductive or thermal energy-conducting configuration or connection with a cooling apparatus 140 configured as a cold generator or in particular as a Peltier element. The functional element 120 is positioned in the immediate vicinity of the gas sensor surface 155 of the gas sensor apparatus 150 configured as a gas sensor chip. The abovementioned elements are placed in the housing 110 that has a contact point, open to gas, to an outer region of the housing 110 in the form of the through-aperture 115.

To sense gases using such a sensor device 100 during operation, the temperature of the functional element 120 is lowered at least below the dew point of a gas to be sensed or of a substance to be detected, using the cooling apparatus 140. A gas flow or fluid flow may in this case be fed either actively or passively to the functional element 120. Dissolved components or components in the form of droplets of the gas or fluid condense on the cooled functional element 120 and form an aqueous phase L or a condensate there. The masses accumulating on the functional element 120 may be detected continuously by measuring a mechanical resonant frequency. It should be borne in mind in this case that an amplitude of the oscillation during the measurement is set such that unintentional atomization or nebulization is avoided. If for example a predefined mass of aqueous phase L is present, water molecules that are held together by hydrogen bridge bonds are torn from their molecular bond by applying a large movement of the functional element 120 and thus converted into the gas phase (atomization or nebulization). The operating frequency during the nebulization does not need to correspond to the measurement frequency in the mass determination. It may be advantageous to excite a higher oscillation mode in the nebulization. According to the exemplary embodiment shown in FIG. 1, the functional element 120 and the gas sensor surface 155 are arranged directly opposite one another.

FIG. 2 shows a schematic sectional illustration of a sensor device 100 according to one exemplary embodiment. In this case, the sensor device 100 corresponds to the sensor device from FIG. 1 with the exception that two through-apertures 115 for a flow of fluid through the housing 110 are formed in the housing 110.

The housing 110 with the through-apertures 115 thus has at least two contact points, open to gas, to the outer region of the housing 110. This has the advantage that defined volumes of gas are able to be channeled through the housing 110 and in particular via the functional element 120 using a gas conveyance system, for example a micropump. This may increase a significance of measurements, since a relationship between analyzed gas volume and measured signals is able to be produced.

FIG. 3 shows a schematic sectional illustration of a sensor device 100 according to one exemplary embodiment. The sensor device 100 is similar to or corresponds to the sensor device from either of the figures described above. In this case, the sensor device 100 corresponds to the sensor device from FIG. 2 with the exception that the functional element 120 is designed as a membrane. According to the exemplary embodiment illustrated in FIG. 3, the membrane is perforated.

The functional element 120 configured as a membrane has the advantage, in combination with the flow configuration from FIG. 2, that the condensate formation or accumulation of the liquid phase L is able to be forced for example on just one side of the membrane. If the functional element 120 is then set in resonance, the generated drops are accelerated onto the other side of the functional element 120. This allows a targeted transfer onto the gas sensor surface 155 and reduces losses in the phase conversion, which results in an increased sensitivity of the sensor device 100.

FIG. 4 shows a schematic sectional illustration of a sensor device 100 according to one exemplary embodiment. The sensor device 100 is similar to or corresponds to the sensor device from one of the figures described above. More precisely, the sensor device 100 corresponds to the sensor device from FIG. 3 with the exception that just one through-aperture 115 is formed in the housing 110 and the housing 110 has a first chamber 411 and a second chamber 412.

The first chamber 411 and the second chamber 412 are partially separated from one another by a separating wall 413 and connected to one another via a connecting aperture 414 or a connecting gap 414. The connecting gap 414 extends between the separating wall 413 and a wall of the housing 110. The functional element 120, the oscillation generator 130 and the cooling apparatus 140 are arranged in the first chamber 411. The gas sensor apparatus 150 is arranged in the second chamber 412. The separating wall 413 is thus arranged between the gas sensor apparatus 150 and the stack consisting of the functional element 120, oscillation generator 130 and cooling apparatus 140.

Both chambers 411 and 412 or both compartments are connected by an aperture, which promotes or facilitates the gas exchange, in the form of the connecting gap 414. This exemplary embodiment has the advantage that the microdroplets generated by way of the oscillation generator 130 do not impinge immediately on the sensor surface 155, but rather first cover a certain path in which the phase transition from liquid to gaseous may take place in an accelerated manner due to an increased surface-to-volume ratio before they impinge on the sensor surface 155. As a result, in particular more components transition into the gas phase, which results in an improved sensitivity of the sensor device 100.

FIG. 5 shows a schematic sectional illustration of a sensor device 100 according to one exemplary embodiment. The sensor device 100 is similar to or corresponds to the sensor device from one of the figures described above. In this case, the sensor device 100 corresponds to the sensor device from FIG. 3 with the exception that just one through-aperture 115 is formed in the housing 110 and the gas sensor apparatus and the cooling apparatus 140 are formed or constructed in or on a common substrate 560 or chip substrate 560.

In other words, the gas sensor chip or the gas sensor apparatus and the cooling apparatus 140 are constructed monolithically on the chip substrate 560. An application-specific integrated circuit (ASIC) for data processing purposes may also additionally be formed or arranged in the chip substrate 560. This has the advantage of a smaller overall structural size, which is advantageous with regard to use in portable devices. In addition, a construction and connection technology is simplified by virtue of fewer process steps, which in turn reduces overall costs of the sensor device 100.

FIG. 6 shows a schematic sectional illustration of a sensor device 100 according to one exemplary embodiment. The sensor device 100 is similar to or corresponds to the sensor device from one of the figures described above. In this case, the sensor device 100 corresponds to the sensor device from FIG. 3 with the exception that the housing 110 has just one through-aperture 115, the sensor device 100 has a capillary element 670 that is arranged spaced apart from the functional element 120 by a capillary intermediate space 675 or capillary gap 675, and a pressure equalization aperture 680 is formed in the housing 110 for pressure equalization between the housing 110 and an environment. In this case, the capillary intermediate space 675 is arranged adjacent to the through-aperture 115.

In this case, the fluid in the form of a liquid to be analyzed comes into contact with the capillary gap 675 through the through-aperture 115, as a result of which the liquid is transported into the housing 110 or sensor housing. The capillary gap 675 is formed by a layer opposite the functional element 120 configured as a membrane, as a capillary element 670 interacting with the functional element 120. If the functional element 120 is set into oscillation, the generated droplets are accelerated in the direction of the sensor surface 155 in a targeted manner and substances that have transitioned into the gas phase are able to be detected by the sensor surface 155. According to this exemplary embodiment, the housing 110 has an additional aperture as pressure equalization aperture 680 in order to create a pressure equalization between the inner housing or housing inner space and the environment, as a result of which the detected gases and generated moisture are able to be channeled out of the housing 110 again.

FIG. 7 shows a schematic sectional illustration of a sensor device 100 according to one exemplary embodiment. The sensor device 100 is similar to or corresponds to the sensor device from one of the figures described above. In this case, the sensor device 100 corresponds the sensor device from FIG. 3 with the exception that the functional element 120 formed as a membrane is clamped on more than one side and separates a region of the two through-apertures 115 from an inner space, containing the gas sensor apparatus 150, of the housing 110, and that a pressure equalization aperture 680 for pressure equalization between the housing 110 and an environment is formed in the housing 110. In this case, the functional element 120 extends between the through-apertures 115 on one side and the pressure equalization aperture 680 and the gas sensor apparatus 150 on the other side.

In other words, the housing 110 has the two through-apertures 115 as inlet and outlet in order to channel liquids over the functional element 120 configured as a membrane at defined flow rates. If the functional element 120 is set into oscillation, partial volumes are continuously drawn from the liquid flow and fed to the gas sensor surface 155. This has the advantage that liquids are able to be continuously analyzed and/or monitored.

FIG. 8 shows a flowchart of a detection method 800 according to one exemplary embodiment. The detection method 800 is able to be executed in order to detect substances in a fluid. In this case, the detection method 800 is able to be executed using or in connection with the sensor device from one of the abovementioned figures or a similar sensor device.

In an actuation step 810, in the detection method 800, the cooling apparatus of the sensor device is actuated so as to cool the functional element in order to condense fluid on the functional element. In this case, an actuation signal is used, for example. The functional element is in particular cooled to a dew point temperature of at least one substance in the fluid. Thereafter, in an excitation step 820, the oscillation generator of the sensor device is excited so as to set the functional element into mechanical oscillations in order to atomize condensed fluid. In this case, an excitation signal is used, for example. Thereafter, in an operating step 830, the gas sensor apparatus is operated or operation thereof is controlled so as to detect gaseous substances in fluid atomized by the functional element. In this case, a sensor signal is in particular received, read and/or evaluated by the gas sensor apparatus.

According to one exemplary embodiment, the detection method 800 also has a step 840 of conveying a fluid flow into the housing of the sensor device and/or through the housing of the sensor device. In this case, the conveyance step 840 is able to be executed continuously. In other words, the actuation step 810, the excitation step 820 and the operating step 830 are able to be executed during an execution of the conveyance step 840.

According to one exemplary embodiment, in the excitation step 820, the oscillation generator is excited so as, in a first quantitative detection mode of operation, to set the functional element into mechanical oscillations at a first amplitude and/or at a first frequency and so as, in a second qualitative detection mode of operation, to set the functional element into mechanical oscillations at a second amplitude and/or at a second frequency. The first amplitude is in this case less than the second amplitude. The first frequency and the second frequency are the same or different.

With reference to the figures described above, exemplary and specific features and/or parameters of exemplary embodiments are explained in more detail below.

The cooling apparatus 140 is for example configured as a Peltier element. Peltier elements are in particular also available in small sizes. For installation in the housing 110, such an element may have for example a footprint of less than or equal to 3 by 3 millimeters squared. A passivation may optionally be provided in order to protect the Peltier element or the cooling apparatus 140 from environmental influences, in particular moisture. By virtue of such passivation, a situation is also able to be prevented whereby materials of the Peltier element, such as for example bismuth, tellurium and the like, are able to affect the sensor function by diffusion. Organic materials, for example parylene, or inorganic materials, such as for example aluminum oxide, silicon oxide, etc., may be used as passivation.

For the oscillation generator 130 or the ultrasound generator, it is for example possible piezoelectric micromachined ultrasonic transducers (cMUTs, pMUTs), piezoelectric films (PVDF=polyvinylidene fluoride), piezoelectric crystals, such as for example lead zirconate titanate (PZT), quartz, lithium niobate, gallium orthophosphate, ferroelectrics, or SAW systems (SAW=surface acoustic wave; typically based on aluminum nitride (AlN)). It may be advantageous likewise to protect the piezoelectric element or the oscillation generator 130 from environmental influences and diffusion by way of a passivation layer.

In one particularly expedient exemplary embodiment, a length of the beam or functional element 120 may be 1 mm, wherein 0.7 mm thereof are free. The width of the functional element 120 may be for example 0.5 mm and the thickness of the functional element 120 may be 20 μm. If holes are intended to be provided in the functional element 120, their diameter, according to one exemplary embodiment, may be 20 μm and/or their distance from one another may be for example 100 μm, wherein the holes are arranged for example in a matrix form. The piezo layer or oscillation generator 130 is for example 5 μm thick, wherein the base (that would sit on the Peltier element) or the cooling apparatus 140 may be for example 0.3 mm high and 0.3 mm wide.

The functional element 120 may be formed for example from steel, steel with a coated surface, for example silver, gold, nickel or another metal that is corrosion-free and possibly able to be soldered, etc. The oscillation generator 130 may be formed for example from lead zirconate titanate. A substrate may be for example formed from silicon or steel or directly be part of the cooling apparatus 140 or a Peltier cooling/heating element.

This results in a resonant frequency of the mechanical oscillations of the functional element of for example around kHz, that is to say outside of the acoustically audible range. A subsequent mode of the resonant oscillation would lie at 207 kHz. Both of the modes mentioned here may be used to measure the accumulated mass by way of the frequency shift, wherein the first mode will exhibit higher sensitivity.

The oscillation generator 130 is constructed in particular by adhesively bonding or soldering a thin steel sheet to a frame or base. A piezo layer may optionally be laminated and/or soldered on beforehand. The functional element 120 and holes are structured for example by lasers, in particular beforehand.

If an exemplary embodiment comprises an “and/or” link between a first feature and a second feature, this should be read such that the first exemplary embodiment has both the first feature and the second feature according to one embodiment and has either just the first feature or just the second feature according to a further embodiment.

1. A sensor device for detecting substances in a fluid, comprising:
a functional element configured to condense and/or to atomize a fluid;
an oscillation generator configured to set the functional element into mechanical oscillations;
a cooling apparatus configured to cool the functional element;
a gas sensor apparatus configured to detect gaseous substances in the fluid atomized by the functional element; and
a housing configured to receive the functional element, the oscillation generator, the cooling apparatus, and the gas sensor apparatus,
wherein at least one through-aperture for the fluid is formed in the housing.

2. The sensor device as claimed in claim 1, wherein the functional element includes a beam or a membrane.

3. The sensor device as claimed in claim 1, wherein the functional element is clamped in the housing on one side.

4. The sensor device as claimed in claim 1, wherein:
the housing has a first chamber and a second chamber,
the first chamber and the second chamber are connected to one another by a connecting aperture,
the functional element, the oscillation generator, and the cooling apparatus are arranged in the first chamber, and
the gas sensor apparatus is arranged in the second chamber.

5. The sensor device as claimed in claim 1, further comprising:
a capillary element arranged spaced apart from the functional element by a capillary intermediate space,
wherein the capillary intermediate space is arranged adjacent to the at least one through-aperture.

6. The sensor device as claimed in claim 1, wherein the at least one through-aperture includes two through-apertures configured to enable a flow of fluid through the housing and/or a pressure equalization aperture is formed for pressure equalization between the housing and an environment.

7. The sensor device as claimed in claim 1, wherein the gas sensor apparatus and the cooling apparatus are formed in and/or on a common substrate.

8. The sensor device as claimed in claim 1, wherein the functional element, the oscillation generator, and the cooling apparatus are arranged in a stacked configuration.

9. A method for detecting substances in a fluid with a sensor device, the method comprising:
actuating a cooling apparatus to cool a functional element in order to condense a fluid on the functional element;
exciting an oscillation generator to set the functional element into mechanical oscillations in order to atomize the condensed fluid; and
operating a gas sensor apparatus to detect gaseous substances in the atomized fluid,
wherein a housing is configured to receive the functional element, the oscillation generator, the cooling apparatus, and the gas sensor apparatus, and
wherein at least one through-aperture for the fluid is formed in the housing.

10. The method as claimed in claim 9, further comprising:
exciting the oscillation generator
in a first quantitative detection mode of operation, to set the functional element into mechanical oscillations at a first amplitude and/or at a first frequency, and
in a second qualitative detection mode of operation, to set the functional element into mechanical oscillations at a second amplitude and/or at a second frequency,
wherein the first amplitude is less than the second amplitude,
wherein the first frequency and the second frequency are the same or different.

11. The method as claimed in claim 9, further comprising:
conveying a fluid flow into the housing of the sensor device and/or through the housing of the sensor device.

12. The method as claimed in claim 9, wherein a controller is configured to execute the method in corresponding units.

13. The method as claimed in claim 9, wherein a computer program is configured to execute the method.

14. The method as claimed in claim 13, wherein the computer program is stored on a machine-readable storage medium.
